SEQUENCE LISTING (11 sequences)

SEQ ID NO: 1 - protein, 7 aa, Artificial
  chemically synthesized; annexin-1 binding compound
  PEPTIDE (1)..(7)
IFLLWQR

SEQ ID NO: 2 - protein, 9 aa, Artificial
  chemically synthesized; annexin-1 binding compound
  PEPTIDE (1)..(9)
IFLLWQRCR

SEQ ID NO: 3 - protein, 11 aa, Artificial
  chemically synthesized; annexin-1 binding compound
  PEPTIDE (1)..(11)
IFLLWQRCRRR

SEQ ID NO: 4 - protein, 10 aa, Artificial
  chemically synthesized; annexin-1 binding compound
  PEPTIDE (1)..(10)
IFLLWQRCRR

SEQ ID NO: 5 - protein, 12 aa, Artificial
  chemically synthesized; annexin-1 binding compound
  PEPTIDE (1)..(12)
IFLLWQRCRRRR

SEQ ID NO: 6 - protein, 8 aa, Artificial
  chemically synthesized; annexin-1 binding compound
  PEPTIDE (1)..(8)
IFLLWQRC

SEQ ID NO: 7 - protein, 7 aa, Artificial
  chemically synthesized; annexin-1 binding compound
  PEPTIDE (1)..(7)
TITWPTM

SEQ ID NO: 8 - protein, 7 aa, Artificial
  chemically synthesized; annexin-1 binding compound
  PEPTIDE (1)..(7)
LRFPTVL

SEQ ID NO: 9 - protein, 7 aa, Artificial
  chemically synthesized; annexin-1 binding compound
  PEPTIDE (1)..(7)
SPTSLLF

SEQ ID NO: 10 - protein, 7 aa, Artificial
  chemically synthesized; annexin-1 binding compound
  PEPTIDE (1)..(7)
MPTLTFR

SEQ ID NO: 11 - protein, 7 aa, Artificial
  chemically synthesized; annexin-1 binding compound
  PEPTIDE (1)..(7)
LLSWPSA

(19) United States
(12) Patent Application Publication
(10) Pub. No.: US 2022/0152204 A1
(43) Pub. Date: May 19, 2022

(54) PHOTOIMMUNOTHERAPY AND PHARMACEUTICAL AGENT USED THEREFOR

(71) Applicant: SHIMADZU CORPORATION, Kyoto-shi, Kyoto (JP)

(72) Inventors: Taka-Aki SATO, Kyoto-shi. Kyoto (JP); Akihiro ISHIKAWA, Kyoto-shi, Kyoto (JP); Masayuki NISHIMURA, Kyoto-shi, Kyoto (JP)

(73) Assignee: SHIMADZU CORPORATION, Kyoto-shi, Kyoto (JP)

(21) Appl. No.: 17/440,461

(22) PCT Filed: Mar. 19, 2020
(86) PCT No.: PCT/US20/23680

§ 371 Date: Sep. 17, 2021

Related U.S. Application Data
(60) Provisional application No. 62/823,803, filed on Mar. 26, 2019.

(51) Int. Cl. A61K 41/00; A61K 47/64; A61P 35/02

(52) U.S. Cl. CPC A61K 41/0071; A61P 35/02; A61K 47/64

(57) ABSTRACT

Provided is a method and pharmaceutical agent which make it possible to efficiently implement PIT, and a method characterized by a step of administering a pharmaceutical agent, in which a substance that binds to tumor blood vessel specific marker molecules present in new blood vessels has been conjugated with at least a labeling substance, to an object associated with a disease or pathology, and changing a physical property of the labeling substance after the administration step.

### CROSS-REFERENCE TO RELATED APPLICATIONS

This application claims priority from U.S. Provisional Application No. 62/823,803, filed Mar. 26, 2019, the disclosure of which is incorporated herein in its entirety by reference.

### TECHNICAL FIELD

The present disclosure relates to photoimmunotherapy and a pharmaceutical agent used therefor.

### BACKGROUND ART

Photoimmunotherapy (PIT), which is a new cancer therapy discovered by Senior Investigator Hisataka Kobayashi et. al. of US National Cancer Institute, uses an antibody conjugated with a chemical substance known as IR700, which is a phthalocyanine derivative (antibody-IR700 conjugate) as a pharmaceutical agent, and is a treatment method with very little side effects, since it does not express toxicity except in cancer cells.

More specifically, this PIT is a photoimmunotherapy which uses IR700 conjugated to an antibody which targets a cell surface protein or to another targeted molecule and activates the IR700 through exposure to near-infrared light in order to enable targeted ablation of specific cells. Using this PIT makes it possible to selectively target disease cells such as tumor cells, thereby making it possible to selectively ablate such cells without injuring healthy cells.

Antibody-IR700 combinations which have been studied to date include, for example, cetuximab-IR700, panitumumab-IR700, zalutumumab-IR700, nimotuzumab-IR700, tositumomab-IR700, rituximab-IR700, ibritumomab tiuxetan-IR700, daclizumab-IR700, gemtuzumab-IR700, alemtuzumab-IR700, CEA-scan Fab fragment-IR700, OC125-IR700, ab75705-IR700, B72.3-IR700, bevacizumab-IR700, basiliximab-IR700, nivolumab-IR700, pembrolizumab-IR700, pidilizumab-IR700, MK-3475-IR700, BMS-936559-IR700, MPDL3280A-IR700, ipilimumab-IR700, tremelimumab-IR700, IMP321-IR700, BMS-986016-IR700, LAG525-IR700, urelumab-IR700, PF-05082566-IR700, TRX518-IR700, MK-4166-IR700, dacetuzumab-IR700, lucatumumab-IR700, SEA-CD40-IR700, CP-870-IR700, CP-893-IR700, MED16469-IR700, MED16383-IR700, MED14736-IR700, MOXR0916-IR700, AMP-224-IR700, PDR001-IR700, MSB0010718C-IR700, rHIgM12B7-IR700, ulocupulumab-IR700, BKT140-IR700, varlilumab−IR700, ARGX-110-IR700, MGA271-IR700, lirilumab-IR700, IPH2201-IR700, AGX-115-IR700, emactuzumab-IR700, CC-90002-IR700 and MNRP1685A-IR700 (patent documents 1, 2, etc.).

### PRIOR ART DOCUMENTS

(Patent document 1)
Published Japanese Translation of a PCT Application 2014-523907
   (Patent document 2)
Published Japanese Translation of a PCT Application 2018-528268

### SUMMARY OF THE INVENTION

### Problem to be Solved by the Invention

PIT is a very effective means that allows one to selectively ablate tumor cells without injuring healthy cells, but it requires that one prepare antibodies which target a cell surface protein.

Here, while studies on antibodies have made much progress, their number is limited and there is a relative lack thereof. Furthermore, when exposure to near-infrared light is performed with pharmaceutical agents in which a specific antibody is conjugated to IR700, the exposure dose differs depending on the type of tumor cell, and improvement toward a more effective PIT which kills a larger number of tumor cells at once is desired.

Furthermore, during growth of cancer tissue, vascular endothelial growth factor (VEGF) 8, fibroblast growth factor (FGF) 9, transforming growth factor (TGF) 1 and the like are released by the cancer cells and by fibroblast cells, epithelial cells and other interstitial cells, inducing the formation of new endothelial cells from nearby blood vessels and thereby leading to the creation of cancer blood vessels. The created blood vessels become a pathway for the supply of nutrients and oxygen to the cancer tissue and also play an essential role in the maintenance of the cancer tissue by supporting the elimination of waste products and the like. Therefore, being able to inhibit the formation of cancer blood vessels may contribute to the development of an effective cancer therapy.

One aspect of the instant disclosure, on the basis of the above-described problem and above-described findings, is to provide a method and pharmaceutical agent which make it possible to more efficiently implement PIT.

### Means of Solving the Problem

Thus, focusing on the new blood vessels induced in the process of malignant tumor growth, one aspect of the disclosure is to employ proteins, peptides or other substances which bind to tumor blood vessel specific marker molecules present in new blood vessels.

Namely, exemplary embodiments include:

a method characterized by a step of administering a pharmaceutical agent, in which a substance that binds to tumor blood vessel specific marker molecules present in new blood vessels has been conjugated with at least a labeling substance, to an object associated with a disease or pathology,

and changing a physical property of the labeling substance after the administration step;

as well as a method wherein the substance which binds to tumor blood vessel specific marker molecules comprises proteins, peptides, aptamers and combinations thereof; and a method characterized in that the physical property of the labeling substance is modified by exposing to radiation, electromagnetic waves or sound waves.

Here, the mechanism of new blood vessels known in the prior art is as follows.

For example, human cells maintain their activity and function by obtaining nutrients and oxygen from blood vessels located in the vicinity of the cell, and the necessary number of cells is strictly controlled by a function inherently possessed by humans. However, cancer cells cannot be controlled and have very active growth, and the cancer cells engaged in such activity require greater amounts of nutrients and oxygen compared to normal cells and thus begin to create new blood vessels. The process of blood vessels being newly formed is called angiogenesis, and such blood vessels are called new blood vessels.

Angiogenesis requires the angiogenic growth factors VEGF (vascular endothelial growth factor) and FGF (fibroblast growth factor), and cancer cells produce these growth factors and destroy the basal lamina of vascular endothelial cells by means of proteolytic enzymes known as matrix metalloproteinases, which stimulate the growth of vascular endothelial cells. The vascular endothelial cells which have been stimulated by the angiogenic growth factors and whereof the basal lamina has been broken then extend new blood vessels to the cancer cells, and the new blood vessels created in this manner reach the cancer cells and become a pipe which feeds nutrients and oxygen to them. Namely, new blood vessels play the role of pathways for infiltration and metastasis of cancer cells, whereby one tumor endothelial cell (TEC) nourishes 100 or more cancer cells, so the death of one TEC means the death of 100 or more cancer cells. This provides a 100 times greater efficiency than with treatment which targets cancer cells.

Therefore, the “object associated with a disease or pathology” of the present disclosure refers to any sort of object in which new blood vessels have been formed, for example, a tumor vascular system, and “disease or pathology” can include, for example, tumors, specifically, cancer.

Furthermore, examples of animals having such a tumor vascular system include, but are not limited to, experimental animals such as mice, rats, hamsters, guinea pigs and rabbits, domestic animals such as pigs, cows, goats and horses, pets such as dogs and cats, primates such as humans, monkeys and chimpanzees, and other mammals.

Furthermore, “administration” means providing or giving the pharmaceutical agent to the subject through any effective route. Examples of administration routes include, but are not limited to, topical, injection (for example, subcutaneous, intramuscular, intradermal, intraperitoneal, intratumoral, intraarterial and intravenous), oral, ocular, sublingual, rectal, percutaneous, intranasal, vaginal and inhalational routes.

Tumor blood vessels specific marker molecules present in new blood vessels include, for example, annexin A1. Annexin A1 is expressed intracellularly in normal cells, but has been reported to be strongly expressed in the lumen walls which are in contact with the endothelial cells of new blood vessels in tumors (Oh et al., Nature 429:629-35 2004), and is an optimal marker molecule for the present disclosure. The exemplary embodiment is however not limited to annexin A1, and the marker molecule may also be selected from a member of the group consisting of annexin A2, annexin A3, annexin A4, annexin A5, annexin A6, annexin A7, annexin A8 and annexin A10.

For example, in proteomics studies which compared the difference in protein quantity between benign and tumor tissue obtained from prostate cancer patients, annexin A3 was identified to be more prevalent in tumors, and the possibility was indicated that it could serve as a diagnostic marker for various subtypes of prostate cancer (for example, Published Japanese Translation of a PCT Application 2010-523990). Furthermore, the migration of annexin A5 to the cell surface is associated with apoptosis.

It should be noted that the exemplary embodiment is not limited to annexin and can employ any marker molecule which is expressed more strongly in new blood vessels than in normal cells.

The term “substances which bind to tumor blood vessel specific marker molecules present in new blood vessels” refers to any substance having the ability to interact with such marker molecules.

For example, compounds such as proteins, peptides and aptamers, which selectively accumulate in tumor blood vessels, or combinations of such compounds, can be used as such substances.

The proteins and peptides would differ depending on the type of marker molecule, but for example, for proteins or peptides which bind to annexin A1, any compound having the ability of interacting with annexin [A]1 can be used. Examples thereof can include the peptides designated IF7 (peptides having the amino acid sequence IFLLWQR (SEQ ID NO: 1)), for instance, the peptides disclosed in Japanese Unexamined Patent Application Publication 2015-110668, namely, IFLLWQRX (IF7-X), IFLLWQRXX (IF7-XX), IFLLWQRXXX (IF7-XXX) and IFLLWQRXXXX (IF7-XXXX), where each X is independently a polar or charged amino acid. For example, each X can be selected from among the entirety of the amino acids C, R, K, S, T, H, D, E, N, Q and M; any set of 10 from the entirety of said amino acids; any set of 9 from the entirety of said amino acids; any set of 8 from the entirety of said amino acids; any set of 7 from the entirety of said amino acids; any set of 6 from the entirety of said amino acids; any set of 5 from the entirety of said amino acids; any set of 4 from the entirety of said amino acids; any set of 3 from the entirety of said amino acids; any set of 2 from the entirety of said amino acids; or any 1 amino acid from among the entirety of said amino acids. For example, each X can be selected independently from a set of three amino acids C, R and K. As another example, each X can be selected independently from a set of two amino acids C and R. In some embodiments, 1 of the aforementioned X's can be C. In some embodiments, 2 of the aforementioned X's can be C. In some embodiments, 2 of the aforementioned X's can be R. In some embodiments, 3 of the aforementioned X's can be R. In some embodiments, 4 of the aforementioned X's can be R. As an example, the aforementioned annexin 1 binding compound can include IFLLWQRCR (SEQ ID NO: 2), IFLLWQRCRR (SEQ ID NO: 3), IFLLWQRCRRR (SEQ ID NO: 4) or IFLLWQRCRRRR (SEQ ID NO: 5).

These peptides which include at least the amino acid sequence IFLLWQR (SEQ ID NO: 1) are referred to collectively as IF7 peptides.

The exemplary embodiment is moreover not limited to IF7 peptides and can also employ the peptides exemplified in WO2018/034356A1, for example, peptides having the amino acid sequence (X1) [D] P [D] (X2) [D] (where X1 represents W or F, X2 represents S or T, and each amino acid sequence number with the symbol [D] appended immediately thereafter represents the D form of the given amino acid), (II) the amino acid sequence P [D] T [D] (X) nF [D] (wherein (X)n represents n of any independently selected amino acids, n represents an integer between 0 and 4, and the symbol [D] has the same meaning as above), or (III) an amino acid sequence which is the retro-inverso of an amino acid sequence of (I) or (II) above, for example, a dTIT7 peptide in which all 7 of the amino acids of TIT7 (7 amino acids starting with threonine-isoleucine-threonine, TITWPTM sequence: SEQ ID NO: 7) are D form amino acids, or a peptide dLRF7, dSPT7, dMPT7 or dLLS7, in which all the amino acids of a peptide with the sequence LRFPTVL (SEQ ID NO: 8), SPTSLLF (SEQ ID NO: 9), MPTLTFR (SEQ ID NO: 10) or LLSWPSA (SEQ ID NO: 11) are D form amino acids.

It is also possible to employ micromolecules or nucleic acid molecules which bind specifically to particular substances known as aptamers. Any compound can be used so long as it binds with tumor blood vessel specific marker molecules and accumulates selectively in tumor blood vessels. Combinations of such compounds may also be used.

The proteins or peptides of the present disclosure can be produced according to known (poly)peptide synthesis methods. The peptide synthesis method can be, for example, a solid phase synthesis method or a liquid phase synthesis method. The desired peptide can be produced by condensing amino acids or a partial peptide capable of forming the peptide of the present disclosure with the remaining portion, and removing the protecting groups if the product contains protecting groups.

A peptide obtained in this manner can be purified and isolated by known purification methods. Here, examples of the purification method include solvent extraction, distillation, column chromatography, liquid chromatography, recrystallization, combinations thereof, etc.

If the peptide obtained by the above method is in free form, the free form can be converted to a suitable salt by known methods or methods based on known methods, and if the peptide is conversely obtained as a salt, the salt can be converted to free form or another salt by known methods or methods based on known methods.

The form of the bonds between the protein or peptide of the present disclosure and one or more components is not particularly limited. The bonds may be direct or indirect via a linker, etc. The bonds may be covalent bonds, non-covalent bonds or a combination thereof. The one or more components may be bonded directly or indirectly to the N terminus, C terminus or other positions of the peptide of the present disclosure. The linkage of a peptide to other components (or a second peptide) is well known in this technical field, and in the conjugate of the present disclosure, this bonding may be accomplished by any known means.

For example, when the bond is via a linker, a known cross-linker (cross-linking agent) such as NHS ester, imido ester, maleimide, carbodiimide, allyl azide, diazirine, isocyanate or psoralen can be used. The peptide of the present disclosure may be modified at one's discretion according to the cross-linker used. For example, cysteine may be added to the C terminus of the peptide of the present disclosure for binding with maleimide linker.

Furthermore, proteins such as albumin may be additionally bound to the linker.

The aforementioned peptide, etc. is conjugated with a labeling substance. The labeling substance can be any substance which can be activated for example by exposing to radiation, electromagnetic waves or sound waves, where radiation includes radiation in the narrow sense, that is, particle radiation such as beta rays, neutron rays, heavy ion rays and meson rays, and electromagnetic radiation such as gamma rays and X-rays. Furthermore, electromagnetic waves include so-called light rays, such as infrared rays, visible light rays and ultraviolet rays, as well as radio waves, and sound waves include ultrasound waves. The term “activated” here signifies that a change in physical properties occurs, such as a change from hydrophilic to hydrophobic, as will be described later.

It should be noted that in the mechanism of cell membrane destruction according to the present disclosure, unlike conventionally known photodynamic therapy (PDT), for example a hydrophilic group of the labeling substance, known as a ligand, is detached, whereby the pharmaceutical agent becomes hydrophobic, and impairment occurs in the cell membrane. Namely, in the present disclosure, a physical property of the labeling substance changes in the state where the labeling substance-peptide conjugate has bonded to a tumor blood vessel specific marker molecule present in a new blood vessel (on a cell membrane), causing membrane-conjugate deformation and aggregate formation and thereby damaging the cancer cell membrane.

In the present disclosure, change of physical properties of the pharmaceutical agent (labeling substance) acts as a “death switch,” and this switch can be turned on by remote control with light which does not exhibit toxicity to the organism, for example, near-infrared light. This is a completely new cell ablation method which allows only pharmaceutical agent which has bonded to cancer cells to be changed to poison by means of light.

In the present disclosure, any substance having the characteristic of becoming a “death switch” as above can be used. However, preferable labeling substances are photosensitive compounds.

A more preferable labeling substance for use in the present disclosure that can be mentioned is phthalocyanine dye.

Phthalocyanines are a group of photosensitizer compounds having a phthalocyanine ring system. Phthalocyanines are azaporphyrins containing four benzoindole groups connected by nitrogen bridges in a 16-member ring of alternating carbon atoms and nitrogen atoms (i.e. C32H16N8), and form stable chelates with metal and non-metal cations. In these compounds, the ring center is occupied by a metal ion (either diamagnetic or paramagnetic) capable of having one or two ligands, depending on the ion. In addition, the periphery of the ring may be either unsubstituted or substituted.

Phthalocyanines strongly absorb red or near-infrared light, with the absorption peak being between approximately 600 nm and 810 nm, and in some cases permit deep penetration of tissue by the light. Phthalocyanines are generally photostable. This photostability is typically advantageous in pigments, dyes and many other applications of phthalocyanines. Phthalocyanine dyes have maximum light absorption in the near-infrared (NIR) range. In some embodiments, the phthalocyanine dye has a maximum light absorption wavelength between 400 nm and 900 nm, for example, between 600 nm and 850 nm, or for example, 680 nm to 850 nm, or for example, about 690±50 nm or 690±20 nm. In some embodiments, the phthalocyanine dye can be efficiently excited with a commercial laser diode that emits light at these wavelength.

In some embodiments, the phthalocyanine dye containing reactive groups is an IR700 NHS ester, for example, an IRDye 700DX NHS ester (Li-Cor 929-70010, 929-70011).

Regarding the means of inducing change in physical properties of the labeling substance, this can be accomplished, for example, by exposure to radiation, electromagnetic waves or sound waves, but is not limited thereto. It can also be accomplished by chemical means.

When using exposure, one can for example expose to a therapeutic dose of radiation or electromagnetic waves with a wavelength in the range of 400 nm to approximately 900 nm or approximately 400 nm to approximately 900 nm, or for example 500 nm to approximately 900 nm or approximately 500 nm to approximately 900 nm, or for example 600 nm to approximately 850 nm or approximately 600 nm to approximately 850 nm, or for example 600 nm to approximately 740 nm or approximately 600 nm to approximately 740 nm, or for example 660 nm to approximately 740 nm, approximately 660 nm to approximately 710 nm, approximately 660 nm to approximately 700 nm, approximately 670 nm to approximately 690 nm, approximately 680 nm to approximately 740 nm, or approximately 690 nm to approximately 710 nm. In some embodiments, cells, for example, tumors, are exposed to a therapeutic dose of radiation or electromagnetic waves of a wavelength between 600 nm and 850 nm, for example, between 660 nm and 740 nm. In some embodiments, cells, for example, tumors, are exposed to a wavelength of at least 600 nm, 620 nm, 640 nm, 660 nm, 680 nm, 700 nm, 720 nm or 740 nm, or approximately at least 600 nm, 620 nm, 640 nm, 660 nm, 680 nm, 700 nm, 720 nm or 740 nm, for example, 690±50 nm or for example 680 nm.

In some embodiments, cells, for example, tumors, are exposed at a dose of at least 1 J/cm², for example, at least 10 J/cm², at least 30 J/cm², at least 50 J/cm², at least 100 J/cm², or at least 500 J/cm². In some embodiments, the exposure dose is 1 to approximately 1,000 or approximately 1 to approximately 1,000 J/cm², approximately 1 to approximately 500 J/cm², approximately 5 to approximately 200 J/cm², approximately 10 to approximately 100 J/cm², or approximately 10 to approximately 50 J/cm². In some embodiments, cells, for example, tumors, are exposed at a dose of at least 2 J/cm², 5 J/cm², 10 J/cm², 25 J/cm², 50 J/cm², 75 J/cm², 100 J/cm², 150 J/cm², 200 J/cm², 300 J/cm², 400 J/cm² or 500 J/cm², or at least approximately 2 J/cm², 5 J/cm², 10 J/cm², 25 J/cm², 50 J/cm², 75 J/cm², 100 J/cm², 150 J/cm², 200 J/cm², 300 J/cm², 400 J/cm² or 500 J/cm².

In some embodiments, cells, for example, tumors, are exposed or illuminated at a dose of at least 1 J/cm fiber length, for example, at least 10 J/cm fiber length, at least 50 J/cm fiber length, at least 100 J/cm fiber length, at least 250 J/cm fiber length or at least 500 J/cm fiber length. In some embodiments, the exposure dose is 1 to approximately 1,000 or approximately 1 to approximately 1,000 J/cm fiber length, approximately 1 to approximately 500 J/cm fiber length, approximately 2 to approximately 500 J/cm fiber length, approximately 50 to approximately 300 J/cm fiber length, approximately 10 to approximately 100 J/cm fiber length, or approximately 10 to approximately 50 J/cm fiber length. In some embodiments, cells, for example, tumors, are exposed to radiation at a dose of at least 2 J/cm fiber length, 5 J/cm fiber length, 10 J/cm fiber length, 25 J/cm fiber length, 50 J/cm fiber length, 75 J/cm fiber length, 100 J/cm fiber length, 150 J/cm fiber length, 200 J/cm fiber length, 250 J/cm fiber length, 300 J/cm fiber length, 400 J/cm fiber length or 500 J/cm fiber length, or at least approximately 2 J/cm fiber length, 5 J/cm fiber length, 10 J/cm fiber length, 25 J/cm fiber length, 50 J/cm fiber length, 75 J/cm fiber length, 100 J/cm fiber length, 150 J/cm fiber length, 200 J/cm fiber length, 250 J/cm fiber length, 300 J/cm fiber length, 400 J/cm fiber length or 500 J/cm fiber length.

In some embodiments, the exposure or illumination dose for a human object is 1 to approximately 400 J/cm² or approximately 1 to approximately 400 J/cm², approximately 2 to approximately 400 J/cm², approximately 1 to approximately 300 J/cm², approximately 10 to approximately 100 J/cm², or approximately 10 to approximately 50 J/cm², for example, at least 10 J/cm² or at least approximately 10 J/cm², or 10 J/cm² or no more than 10 J/cm², or no more than approximately 10 J/cm², or 10 J/cm², or approximately 10 J/cm², at least 30 J/cm², at least 50 J/cm², or at least 100 J/cm². In some embodiments, the exposure or illumination dose for a human object is 1 to 300 J/cm fiber length or approximately 1 to 300 J/cm fiber length, 10 to 100 J/cm fiber length or approximately 10 to 100 J/cm fiber length, or else 10 to 50 J/cm fiber length or approximately 10 to 50 J/cm fiber length, for example, at least 10 J/cm fiber length or at least approximately 10 J/cm fiber length, or else 10 J/cm fiber length or no more than 10 J/cm fiber length or no more than approximately 10 J/cm fiber length, or else 10 J/cm fiber length, or else approximately 10 J/cm fiber length, at least 30 J/cm fiber length, at least 50 J/cm fiber length, or at least 100 J/cm fiber length. In some cases, the exposure dose for achieving PIT of a human object is less than the dose required for PIT in mice.

In the present disclosure, multiple dyes may be conjugated. For the second dye, a second dye is selected which provides better fluorescence through visualization than the first dye (for example, IR700). The second dye is used both for fluorescent imaging and PIT. For example, exposing the lesion or tumor achieves detection of the presence of conjugate in the lesion or tumor in the object of treatment by causing a fluorescent signal to be radiated from the second fluorescent dye. In some embodiments, using a conjugate, binding of the dye to the target site (for example, a tumor) can be monitored through fluorescent imaging of the second dye, and cells associated with the disease or pathology, for example, tumor cells, can be eradicated using photoimmunotherapy based on activation of the first dye (for example IR700).

The second dye can be, for example, hydroxycoumarin, Cascade Blue, Dylight 405 Pacific Orange, Alexa Fluor 430, fluorescein, Oregon Green, Alexa Fluor 488, BODIPY 493, 2,7-dichlorofluorescein, ATTO 488, Chromeo 488, Dylight 488, HiLyte 488, Alexa Fluor 532, Alexa Fluor 555, ATTO 550, BODIPY TMR-X, CF 555, Chromeo 546, Cy3, TMR, TRITC, Dy547, Dy548, Dy549, HiLyte 555, Dylight 550, BODIPY 564, Alexa Fluor 568, Alexa Fluor 594, rhodamine, Texas Red, Alexa Fluor 610, Alexa Fluor 633, Dylight 633, Alexa Fluor 647, APC, ATTO 655, CF633, CF640R, Chromeo 642, Cy5, Dylight 650, Alexa Fluor 680, IRDye 680, Alexa Fluor 700, Cy5.5, ICG, Alexa Fluor 750, Dylight 755, IRDye 750, Cy7, Cy7.5, Alexa Fluor 790, Dylight 800, IRDye 800, Qdot (registered trademark) 525, Qdot (registered trademark) 565, Qdot (registered trademark) 605, Qdot (registered trademark) 655, Qdot (registered trademark) 705, or Qdot (registered trademark) 800.

Furthermore, in the present disclosure, the pharmaceutical agent may include a therapeutic agent, examples of which include anticancer agents, molecular targeted drugs, hormonal agents and immunostimulants.

As the anticancer agents, known anticancer agents can be used, such as “antimetabolites,” which suppress the growth of cancer cells, “alkylating agents,” which damage the DNA of cancer cells, “anticancer antibiotics,” which destroy cancer cell membranes and suppress synthesis of cancer DNA, “microtubule-acting drugs,” which act by stopping the operation of microtubules, “platinum formulations,” which suppress cancer cell division by binding to DNA, “topoisomerase inhibitors,” which act by suppressing the operation of enzymes for synthesizing DNA, and the like.

The antimetabolites can be, for example, antifolate drugs, dihydropteroate synthase inhibitors, dihydrofolate reductase inhibitors (DHFR inhibitors), pyrimidine metabolism inhibitors, thymidylate synthase inhibitors, purine metabolism inhibitors, IMPDH inhibitors, ribonucleotide reductase inhibitors, nucleotide analogs, L-asparaginase, etc. Specific examples of antimetabolites include enositabine (Sunrabin), capecitabine (Xeloda), carmofur (Mifurol), cladribine (Leustatin), gemcitabine (Gemzar), cytarabine (Cylocide), cytarabine ocfosfate (Starasid), tegafur (Atilon, Aftoful, Tefsiel, Futraful, Lunacin, etc.), tegafur-uracil (UFT), tegafur-gimeracil-oteracil potassium (TS-1), doxifluridine (Furtulon), nelarabine (Arranon G), hydroxycarbamide (Hydrea), fluorouracil (5-FU, Carzonal, Bennan, Lunachol, Lunapon), fludarabine (Fludara), pemetrexed (Alimta), pentostatin (Coforin), mercaptopurine (Leukerin), methotrexate (Methotrexate), etc.

Specific examples of alkylating agents include cyclophosphamide (Endoxan), ifosfamide (Ifomide), melphalan (Alkeran), busulfan, thiotepa (Tespamin) and other nitrogen mustard based alkylating agents; nimustine (Nidran), ranimustine (Cymerin), dacarbazine (Dacarbazine), procarbazine (Procarbazine Hydrochloride), temozolomide (Temodar), carmustine (Gliadel), streptozotocin (Zanosar), bendamustine (Treakisym) and other nitrosourea based alkylating agents, etc.

Specific examples of anticancer antibiotics include actinomycin D (Cosmegen), aclarubicin (Aclacinon), amrubicin (Called), idarubicin (Idamycin), epirubicin (Epirubicin Hydrochloride, Farmorubicin), zinostatin stimalamer (SMANCS), daunorubicin (Daunomycin), doxorubicin (Adriacin), pirarubicin (Pinorubin, Therarubicin), bleomycin (Bleo), peplomycin (Pepleo), mitomycin C (Mitomycin), mitoxantrone (Novantrone), liposomal doxorubicin (Doxil), etc.

Examples of microtubule inhibitors include vinblastine (Exal), vincristine (Oncovin), vindesine (Fildesine) and other vinca alkaloid based microtubule polymerization inhibitors; paclitaxel (Taxol), docetaxel (Taxotere) and other taxane based microtubule depolymerization inhibitors, etc.

Examples of platinum formulations include oxaliplatin (Elplat), carboplatin (Carboplatin, Carbomerck, Paraplatin), cisplatin (IA-call, Conabri, Cisplatin, etc.), nedaplatin (Aqupla), etc.

Examples of topoisomerase inhibitors include camptothecin and derivatives thereof (for example, irinotecan (Campto), nogitecan (Hycamtin), SN-38, etc.) and other type I topoisomerase inhibitors; doxorubicin (Adriacin) and other anthracycline based drugs, etoposide (Lastet, Vepesid) and other epipodophyllotoxin based drugs, levofloxacin (Cravit), ciprofloxacin (Ciproxan) and other quinolone based drugs, and other type II topoisomerase inhibitors.

Furthermore, “molecular targeted drugs” are typically drugs which target a protein known as epidermal growth factor receptor (EGFR), which is present in large amounts on the surface of cancer cells and is involved in cell proliferation; molecular targeted drugs which target EGFR are known to have characteristic side effects such as skin disorders, and it is important to skillfully prevent these while carrying out treatment.

Other examples include molecular targeted drugs which target molecules such as HER2, ALK, ROS1, mTOR, CDK4/6, BCR-Abl, CCR4 and VEGF.

Specific examples of molecular targeted drugs include regorafenib (Stivarga), cetuximab (Erbitux), panitumumab (Vectibix), ramucirumab (Cyramza), gefitinib (Iressa), erlotinib (Tarceva), afatinib (Giotrif), crizotinib (Xalkori), alectinib (Alecensa), ceritinib, lenvatinib (Lenvima), trastuzumab (Herceptin), lapatinib (Tykerb), pertuzumab (Perjeta), sunitinib (Sutent), sorafenib (Nexavar), axitinib (Inlyta), pazopanib (Votrient), nivolumab (Opdivo), pembrolizumab, ipilimumab (Yervoy), vemurafenib (Zelboraf), everolimus (Afinitor), temsirolimus (Torisel), rituximab (Rituxan), bevacizumab (Avastin), geldanamycin, etc.

### Effect of the Invention

According to the present disclosure, new blood vessels are targeted, and since one tumor endothelial cell (TEC) nourishes 100 or more cancer cells, the death of one TEC means the death of 100 or more cancer cells. Therefore, according to the present disclosure, the treatment is 100 times more efficient than treatment targeting cancer cells.

Namely, if the construction of cancer blood vessels is inhibited, cancer cells can be starved out, which may provide for an extremely efficient cancer therapy. Furthermore, cancer blood vessels may constitute common tumor-associated antigens (TAAs) independent of the cancer type. While cancer cells generally have different characteristics depending on the organ in which they arise, cancer blood vessels are constructed from TECs based on the host's vascular endothelial cells, and are thus expected to have the same TAAs regardless of the type of cancerous organ. The inhibition of angiogenesis in cancer tissue can not only effectively suppress growth by interrupting the nutrient supply pathway to the cancer tissue cells, but can also be expected to become a highly universal therapy which can be applied to all sorts of cancers.

### BRIEF DESCRIPTION OF THE DRAWINGS

FIG. 1 is a drawing illustrating the conjugation of IF7-C and IR700.

FIG. 2 illustrates mechanism of cell membrane destruction according to the present disclosure.

FIG. 3 shows (a) Reaction diagram of maleimidation of IR700, and (b) Diagram of composite analyzed by RPLC.

FIG. 4 is a reaction diagram of conjugation of IFC-7 and IR700 maleimide.

FIG. 5 shows fluorescent micrographs.

### EMBODIMENT OF THE INVENTION

### Embodiment Example 1

### Conjugation of IF7-C and IR700

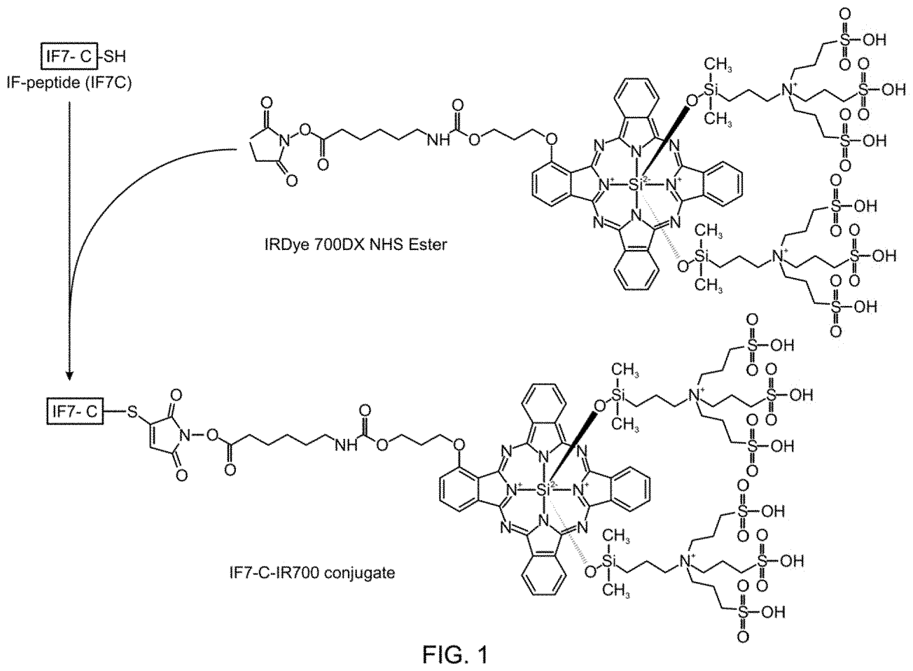

FIG. 1 illustrates the conjugation of IF7-C peptide (peptide having the amino acid sequence IFLLWQRC: SEQ ID NO: 6) and IR700.

IF7-C is synthesized using known techniques, namely either Fmoc or Boc chemistry, on commercial synthesis equipment.

Furthermore, IR700 (IRDye 700) is sold by Li-Cor, and can be covalently bonded with IF7-C using an NHS ester of IR700 (IRDye 700DX NHS ester: Li-Cor 929-70010, 929-70011).

Specifically, in order to bond IF7-C peptide with IRDye 700DX NHS ester, the two were dissolved in methanol at a molar ratio of 1:1. An equal volume of pure water was added to the mixture and left for 2 hours at room temperature. The product was purified using a C18 reverse phase HPLC column (10×150 mm) by means of gradient elution with 40% to 50% acetonitrile in water containing 0.1% (v/v) trifluoroacetic acid at a flow rate of 2.5 ml/min. The purity and structure of IF7-C peptide and IRDye 700DX were evaluated through ESI mass spectrometry.

### Exposure of Cells

For purposes of PIT, cells were inoculated onto a coverglass dish with a 35 mm bottom and incubated for 24 hours. The medium was replaced with fresh culture medium containing IF7-C peptide-IRDye 700DX at 10 μg/mL and incubated for 6 hours at 37° C. After washing with phosphate buffer solution (PBS), the culture medium was replaced with phenol red-free medium. Using a red light emitting diode (LED; FluorVivo; INDEC Systems Inc., Capitola, Calif.), the cells were exposed to light of 670 nm to 690 nm at an output density of 2.6 mW/cm² as measured with an optical power meter (PM 100, Thorlabs, Newton, N.J.). The cell survival rate was evaluated after 1 hour of treatment using a LIVE/DEAD (registered trademark) Fixable Green Dead Cell Stain Kit (Invitrogen). After the treatment, the cells were trypsinized and washed with PBS. A green fluorescent reactive dye was added to the cell suspension, incubating for 30 minutes at room temperature. Next, the cells were analyzed with a flow cytometer (FACS Calibur, BD BioSciences, San Jose, Calif.).

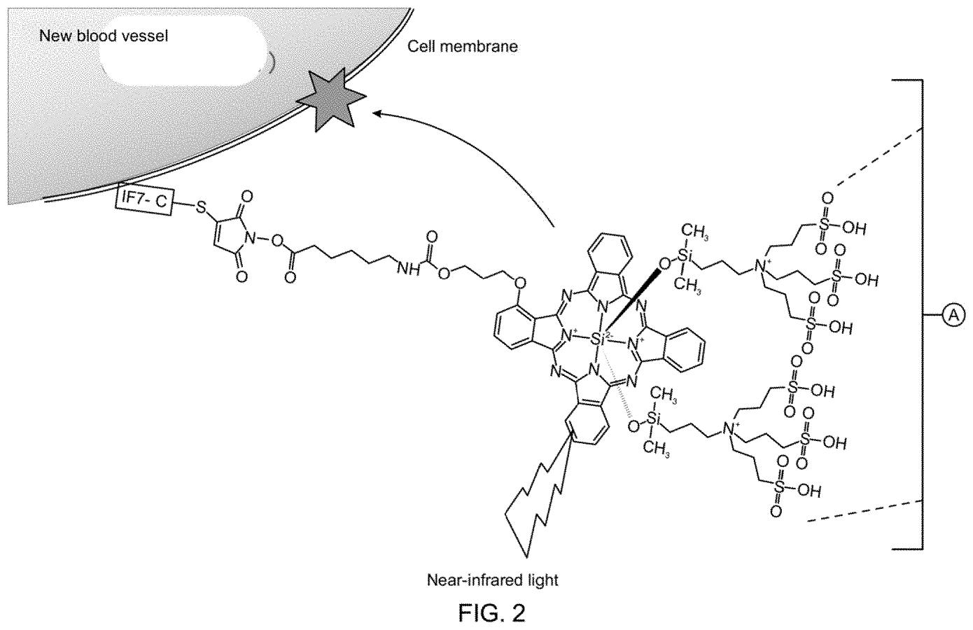

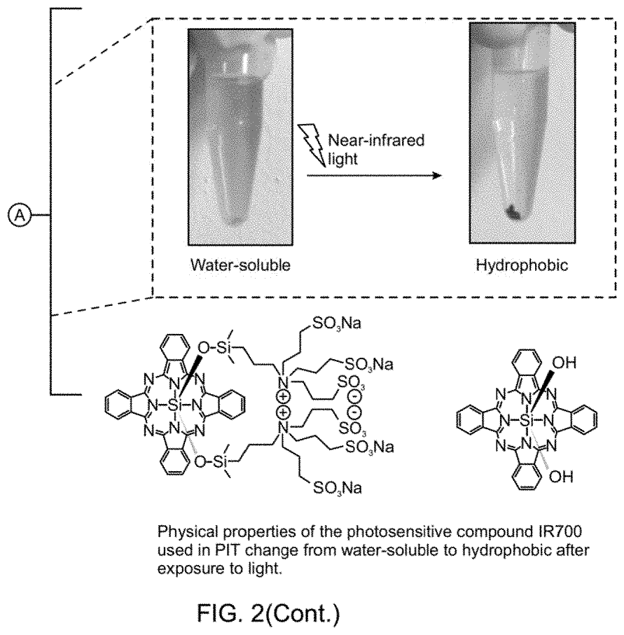

The mechanism of cell membrane destruction according to the present disclosure is illustrated in FIG. 2.

The conjugate of IF7-C and IR700 binds with annexin A1 of new blood vessels. Upon exposure to near-infrared light, the physical properties of IR700 change, after the light exposure, from water-soluble to hydrophobic. The change in chemical structure of IR700 induces a change in the steric structure of IF7-C, causing damage to the cell membrane.

The near-infrared exposure dose is preferably less than 10 J/cm² so as not to injure the new blood vessels, which is lower than the dose used in conventional PIT, but the dose can be selected at one's discretion.

### Embodiment Example 2

### Maleimidation of IR700

NHS was replaced with maleimide in order to enable IR700 to efficiently conjugate to the cysteine SH residue of IF7-C.

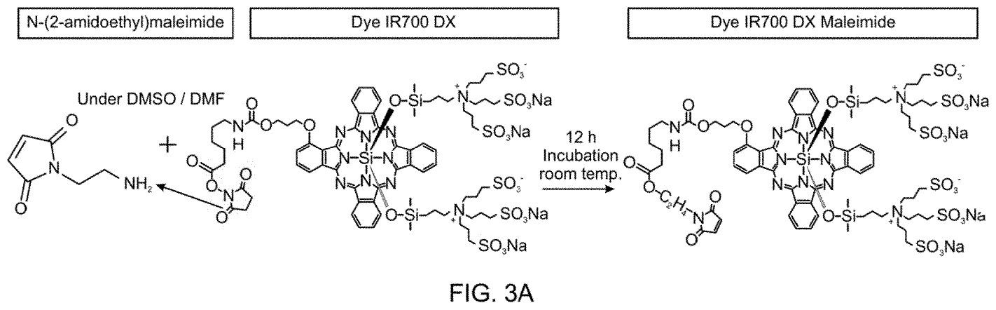

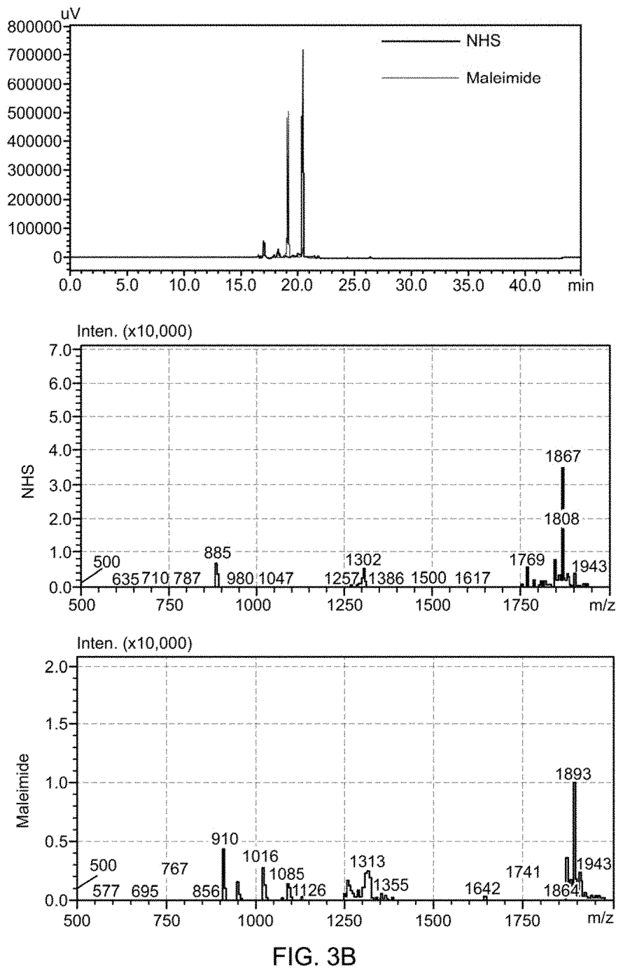

Regarding the synthesis technique, as shown in FIG. 3 (a), when the synthesized product was verified by RPLC, a maleimidated IR700 peak and a minimum amount of unreacted IR700 were confirmed from the synthesis product (FIG. 3 (b)).

### Conjugation of IFC-7 and IR700 Maleimide

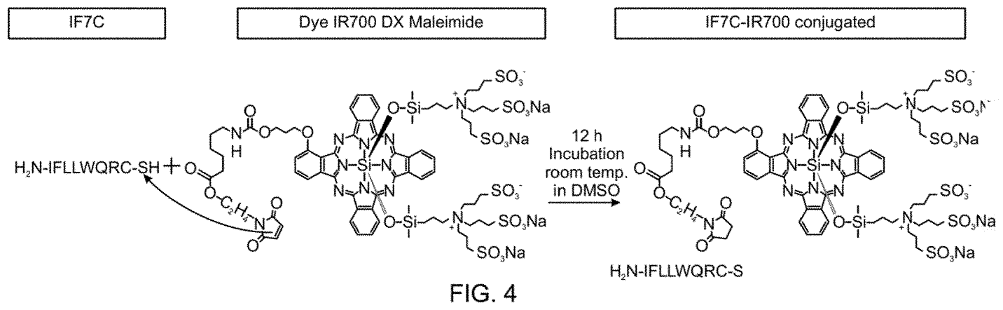

IF7-C was conjugated with IR700 maleimide. After synthesizing as shown in FIG. 4, the product was separated through RPLC and recovered by evaporating and drying.

### Observation Under Fluorescent Microscope

Epidermal cancer cells (A431) were placed at 10,000 cells/mL into a 3.5 mm dish and incubated for 1 day. Subsequently, 20 μg of IF7C-IR700 or IR700 was added to the respective dish and incubation was performed for 10 minutes at 37° C.

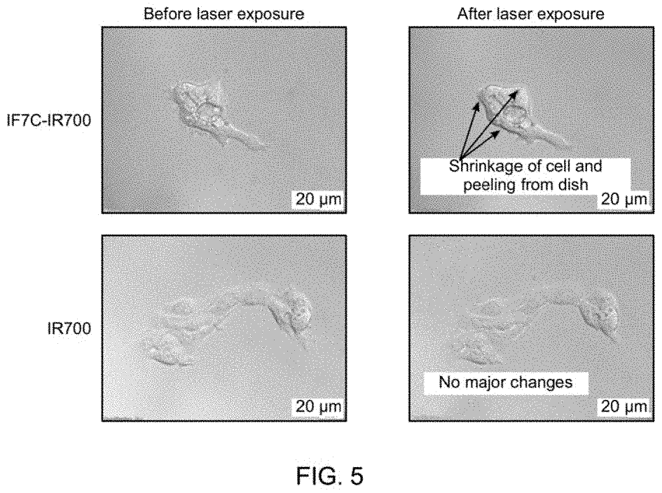

Exposure to a 690 nm laser was performed at 50 J, and the A431 cells before and after exposure were observed under a fluorescent microscope (IX61 or IX81; Olympus America). The observation results, as shown in FIG. 5, were that in dishes to which only IF700 had been added, the cell did not shrink, while in the dish to which IF7C-IR700 had been added, the cells had shrunk. From this, it can be inferred that IF7C-IR700 is internalized by cells and undergoes a reaction, causing the cells to shrink.

### Mouse Experiment

Five xenograft model animals implanted with A431 were treated with 0.033 μmol IF7C-IR700, and the fluorescent intensity of body surface, tumor and liver of the mice was monitored at 10 min intervals for up to 60 min and at 30 min intervals for up to 180 min with a Pearl Imager (LI-COR Bioscience) to check for accumulation of pharmaceutical agent. The fluorescent intensity over the tumor reached a maximum at 60 min, so exposure to therapeutic light was performed 60 min after pharmaceutical agent injection to carry out NIR-PIT with IF7C-IR700. For the PIT, exposure to NIR light was performed under conditions which enable cleavage of the IR700, at a wavelength of 680 nm to 690 nm and a dose of 10 J/cm².

With the pharmaceutical agent of the present disclosure, pharmaceutical agent accumulation is rapid and PIT is possible in about several tens of minutes to one hour after pharmaceutical agent administration. This is advantageous compared to conventional methods, in which pharmaceutical agent accumulation took a long time and 1 to 2 days would be required after pharmaceutical agent administration until PIT.

1. A method characterized by a step of administering a pharmaceutical agent, in which a substance that binds to tumor blood vessel specific marker molecules present in new blood vessels has been conjugated with at least a labeling substance, to an object associated with a disease or pathology,
and changing a physical property of the labeling substance after the administration step.

2. The method as set forth in claim 1, wherein the substance which binds to tumor blood vessel specific marker molecules is selected from the group consisting of proteins, peptides, aptamers and combinations thereof.

3. The method as set forth in claim 1, characterized in that the physical property of the labeling substance is modified by exposing to radiation, electromagnetic waves or sound waves.

4. The method as set forth in claim 1, wherein the tumor blood vessel specific marker molecule is any of annexin A1, annexin A2, annexin A3, annexin A4, annexin A5, annexin A6, annexin A7, annexin A8 and annexin A10.

5. The method wherein the peptide set forth in claim 2 is a peptide including at least the amino acid sequence of SEQ ID NO: 1, or a dTIT7 peptide which all of the amino acids of SEQ ID NO: 7 are D form amino acids.

6. The method as set forth in claim 5, wherein the peptide including at least the amino acid sequence of SEQ ID NO: 1 is an IF7 peptide.

7. The method as set forth in claim 1, wherein the object associated with a disease or pathology is a tumor blood vessel system.

8. The method as set forth in claim 1, wherein the labeling substance is a substance activated by exposure to radiation or electromagnetic waves or sound waves.

9. The method as set forth in claim 8, wherein the electromagnetic waves are near-infrared light, and the substance activated by exposure to near-infrared light is a phthalocyanine dye.

10. The method as set forth in claim 9, wherein the phthalocyanine dye is IR700.

11. The method as set forth in claim 1, characterized in that the pharmaceutical agent contains a therapeutic agent.

12. The method as set forth in claim 11, wherein the therapeutic agent is selected from among anticancer agents, molecular target drugs, hormonal agents and immunostimulants.

13. A pharmaceutical agent in which a substance that binds to tumor blood vessel specific marker molecules present in new blood vessels has been conjugated with at least a labeling substance.

14. The pharmaceutical agent as set forth in claim 13, wherein the substance which binds to tumor blood vessel specific marker molecules is selected from the group consisting of proteins, peptides, aptamers and combinations thereof.

15. The pharmaceutical agent as set forth in claim 14, characterized in that it contains two or more types of labeling substances.

16. The pharmaceutical agent as set forth in claim 14, additionally containing a therapeutic agent.

17. The pharmaceutical agent wherein the IF7 peptide has been conjugated with IR700.

18. The pharmaceutical agent as set forth in claim 17, wherein the IF7 peptide has been conjugated via a linker with IR700.

19. The pharmaceutical agent as set forth in claim 18, wherein the linker comprises any of NHS ester, imide ester, maleimide, carbodiimide, allyl azide, diazirine, isocyanate or psoralen.

20. The pharmaceutical agent as set forth in claim 19, wherein albumin is additionally bonded to the linker
